(19) United States
(12) Patent Application Publication
(10) Pub. No.: US 2006/0232272 A1
(43) Pub. Date: Oct. 19, 2006

(54) Imaging apparatus and method

(75) Inventor: Peter Hanley, GLOUCESTERSHIRE (GB)

Correspondence Address: STAAS & HALSEY LLP, SUITE 700, 1201 NEW YORK AVENUE, N.W., WASHINGTON, DC, 20005, US

(73) Assignee: OXFORD INSTRUMENTS, EYNSHAM, WITNEY, OXON, (GB)

(21) Appl. No.: 10/485,959

(22) PCT Filed: Aug. 12, 2002
(86) PCT No.: PCT/GB02/03700

§ 371 Date: Jun. 30, 2004

(30) Foreign Application Priority Data
Aug. 14, 2001 (GB) 0119800.1

(51) Int. Cl. G01V 3/00; A61B 5/05

(52) U.S. Cl. 324/309; 600/410

(57) ABSTRACT

A method of monitoring a surgical procedure, the method comprising exposing a region of interest to a static magnetic field with sufficient uniformity to carry out a magnetic resonance process; exposing the region of interest to a RF magnetic field having at least one gradient and detecting magnetic resonance signals emitted from the region of interest; and generating an image of at least one feature in the region of interest from the received signals.

The invention relates to an imaging apparatus and a method for monitoring a surgical, particularly a minimally invasive surgical, procedure.

A class of surgical procedures including catheterisation, endoscopy, balloon angioplasty, keyhole surgery etc are regarded as being minimally invasive and hence less traumatic than more open techniques. Applications include the repair of aneurisms, the removal of obstructions in blood vessels and the taking of biopsies from internal organs and tissues.

Traditionally, guidance is by the surgeon manipulating the catheter and the process is monitored using either ultrasound imaging or X-ray fluoroscopy. Ultrasound suffers from poor spatial resolution and the images are difficult to interpret. X-rays, because of the need for nearly continuous monitoring, involve large radiation doses both for the patient and also for the operator. For this reason, there is growing interest in “interventional MRI” where magnetic resonance imaging is used for monitoring the procedure.

Central to the practicality of interventional MRI is the question of access: traditional MRI instruments which rely on switched B₀ field gradients either involve inserting the patient into a narrow tube formed by the magnet and gradient coils, or into the gap between the poles of a “C-magnet”. In cases where the magnet can be designed to improve access, the gradient coils undo some of these advantages, or in the case of the “Double Donut” magnet are extremely complex and expensive. See, for example, “Design of a Mid-Field Intraoperative MR System at 0.5 Tesla”, Hushek SG, Interventional MRI, ed Lufkin, Mosby (1999). A further disadvantage is that when the patient is lightly sedated, the noise resulting from the switched gradients can lead to involuntary movement.

In accordance with the present invention, a method of monitoring a surgical procedure comprises exposing a region of interest to a static magnetic field with sufficient uniformity to carry out a magnetic resonance process; exposing the region of interest to a RF magnetic field having at least one gradient and detecting magnetic resonance signals emitted from the region of interest; and generating an image of at least one feature in the region of interest from the received signals.

With this invention, the need for switched field gradient coils is removed and instead the RF field is used to achieve spatial imaging. These techniques are similar to those known as “rotating-frame imaging” (RFI) or “rotating frame zeugmatography” described in more detail in “Rotating Frame Zeugmatography”, Hoult D I, J. Magn. Reson. 33, 183-197 (1979) and “Rotating Frame Spectroscopy and Spectroscopic Imaging”, Styles P, NMR Basic Principles and Progress 27, Springer-Verlag (1992). These techniques have generally been used for diffusion studies or for spatially resolved NMR spectroscopy. The reasons for this are that imaging using B₀ field gradients is generally more versatile and that the processing of the received signal to produce the image is relatively straightforward. By comparison, the signal arising from RFI is the result of non-linear processes and contains “off-resonance” components. However, mathematical techniques have been developed to deal with these. See “A Parallel Algorithm for Rotating Frame Zeugmatography”, Chen C-N, Hoult D I and Sank V J, Mag. Reson Med 1 3, 354 (1984), “Maximum Entropy Reconstruction of Rotating Frame Zeugmatography Data”, Hore P J and Daniell G J, J. Magn. Reson., 69, 386-390 (1986), and “Suppression of Artifacts in the Phase-Modulated Rotating Frame Imaging Experiment Using the Maximum Entropy Method”, Jones J A, Hore P J, Relf C P, Ouwerkerk R, and Styles P, J. Magn. Reson., 98, 73-80 (1992). Further, with the advent of cheap but powerful computing, this is no longer a significant drawback.

In the context of interventional MRI, RFI has a significant advantage in that it is not limited by the settling time of switched gradients (a consequence of eddy currents induced in the magnet and other surroundings) and so is able to receive signal from materials with short T₂ (transverse relaxation time), see “An Assessment of Spin-Echo Rotating-Frame Imaging for Spatially Localized Determination of Short T₂ Relaxation Times in Vivo”, Dixon R M and Styles P, Proc 10^{th} Annual Meeting Soc Magn Reson Med, San Francisco (1991). This enables not only bone to be imaged, but also polymer materials from which catheters might be made. Thus it is possible to devise pulse sequences which would enable a catheter to be highlighted on an image of the surrounding tissue. In contrast to this, conventional field-gradient imaging can only distinguish such materials by a null signal (which might not be visible when the slice thickness is greater than the size of the instrument) or by means of antennae attached to the instrument.

A further advantage of RFI for this-application, is that the spatial encoding for one or two of the dimensions is carried by means of gradients applied to the radio-frequency transmitter field, and so by attaching the relatively small and light transmitter coils to the patient, problems of patient movement during imaging are much reduced.

The invention includes the application of rotating frame imaging to the monitoring of minimally invasive surgical procedures, of producing two-dimensional images of a selected slice during such procedures, of highlighting polymer and other non-metallic materials used in the instruments such as catheters employed in minimally-invasive surgery by virtue of their transverse relaxation time and also of performing such imaging when the instruments are guided by an applied magnetic field.

In this context, the method can be used for monitoring the location of a catheter. This could then be extended to the use of the magnetic field generating system to steer the catheter. (See WO02/43797).

Three examples of apparatus and methods according to the invention will now be described with reference to FIGS. 1 to 3 which are schematic views of the apparatus of each example respectively.

In FIG. 1, a main, strong, relatively homogeneous field B₀ is provided by a set of counter-wound coils 1,2 with a static, uniform gradient of the form

$$\frac{\partial {B}_{z}}{\partial z}$$

superimposed upon it. This magnet might instead be a solenoid, a split-coil-pair, an iron-cored C-magnet, an iron-cored window-frame magnet, or any other arrangement as might be suitable for the procedure. The gradient might be provided by an imbalance between the coils of a split pair, or C-magnet, for example, or by an additional coil. In addition, an RF coil system is provided including a B₁₋ₓ coil consisting of two elements 4. Using one element 4 only, or both elements 4 series aiding it, produces a gradient of the form

$$\frac{\partial {B}_{1 x}}{\partial x} .$$

With both coils 4 connected in series opposition, the radii and relative number of turns on each are chosen to as to produce a relatively uniform B₁ field at the centre of the region of interest 3. Another RF coil 5 produces a gradient of the form

$$\frac{\partial {B}_{1 y}}{\partial y} .$$

With this system, the x and y RF coils could he used to receive with both coils, and their signals combined in quadrature to improve the signal-to-noise ratio by a factor of \sqrt{2}. Alternatively, a separate receiver coil could be used, with isolation between transmitter and receiver being achieved electronically, as has been described in the literature.

In use, a patient will be located suitably with respect to the region of interest 3 so that a minimally invasive surgical procedure can be monitored and imaged. Indeed, it is conceivable that the radio frequency coils 4,5 could be attached to the patient himself.

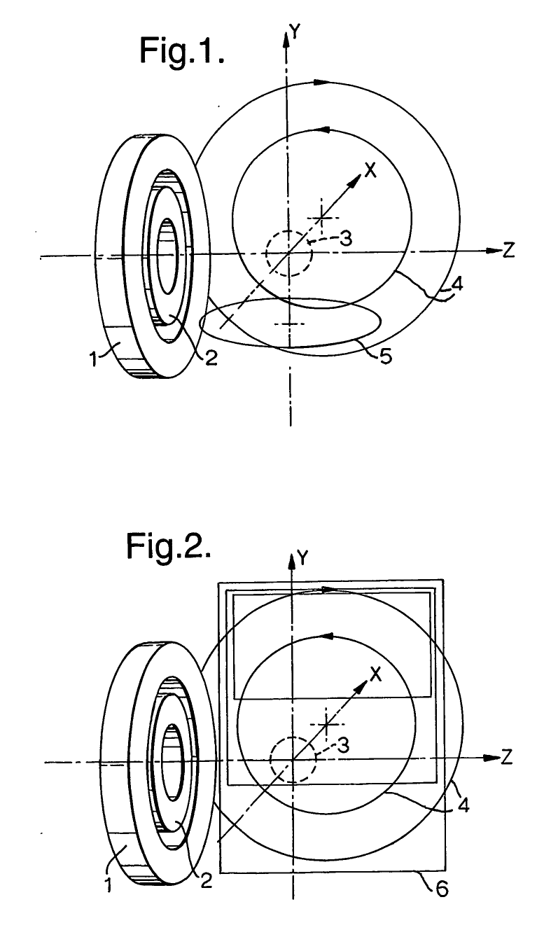

FIG. 2 shows an example of a set of RF coils of a second configuration. In this case, the B₁ₓ coils 4 are the same as in the previous example, capable of producing a gradient of the form

$$\frac{\partial {B}_{1 x}}{\partial x}$$

or a relatively uniform B₁ field. The B_{1y} coil 6 is similar to that described in “Single Coil Surface Imaging Using a Radiofrequency Field Gradient”, Baril N, Thiaudière E, Quesson B, Delalande C, Canioni P and Franconi J-M, J Magn Reson, 146, 221-227 (2000) and produces a gradient of the form

$$\frac{\partial {B}_{1 y}}{\partial y} .$$

With this apparatus, it is possible to produce a two-dimensional image from a selected slice in the following way:

A slice can be selected in the Y-Z plane using a selective excitation scheme such as described in “The Technique of Rotating Frame Selective Excitation and Some Experimental Results”, Hedges L K and Hoult D I, J Magn Reson, 79, 361-403 (1988) or “Accurate Spatial Localization by a Novel Sequence Using a RF Field Gradient and a DANTE-like Pulse Train”, Canet D, Boudot D, Belmajdoub A, Retournard A and Brondeau J, J Magn Reson, 79, 168-175 (1998). A series of refocusing pulses are then applied using the B_{1y} gradient, with successive pulses being incremented, and the echo resulting from each being acquired.

At its simplest, data processing can consist of a two-dimensional Fourier transform: The B₀ gradient provides spatial encoding via frequency in the Z-direction and the B_{1y} gradient supplies spatial encoding via phase in the Y-direction. Selection has already taken place in the X-direction. In practice, the signal will be confused by off-resonance effects, and more complex signal processing will be required, such as that described in “A Parallel Algorithm for Rotating Frame Zeugmatography”, Chen C-N, Hoult D I and Sank V J, Mag. Reson Med 1 3, 354 (1984), “Maximum Entropy Reconstruction of Rotating Frame Zeugmatography Data”, Hore P J and Daniell G J, J. Magn. Reson., 69, 386-390 (1986), and “Suppression of Artifacts in the Phase-Modulated Rotating Frame Imagingexperiment Using the Maximum Entropy Method”, Jones J A, Hore P J, Relf C P, Ouwerkerk R, and Styles P, J. Magn. Reson., 98, 73-80 (1992).

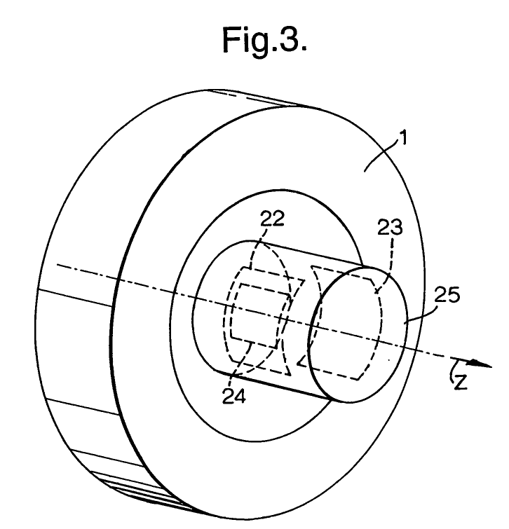

FIG. 3 illustrates an alternative arrangement utilizing a combination of rotating frame and projection reconstruction. As before, the apparatus comprises a main magnet 1 which generates a static B0 magnetic field with a static gradient in the Z-direction.

A pair of RF coils 22,23 produce a uniform RF field for refocussing pulses while the rf coil 23 in combination with an RF coil 24 together produce a gradient RF field for “rotating frame” imaging. Thus, a static B0 gradient is provided in the Z-direction and a RF B1 gradient in the radial direction.

In use, rotating frame imaging (RFZ) allows a spin-density map in the r-z plane to be obtained. If B1 is then rotated about the Z-axis in steps, a set of data is obtained which can be used to create a three-dimensional image using projection-reconstruction. The rotation could be done electrically or mechanically. The cylindrical surface swept out by rotation of the radio frequency coils 22-24 is shown at 25 in FIG. 3. This process is described in more detail in Jones et al, J. Mag Res 98, 73-80 (1992).

1. A method of monitoring a surgical procedure, the method comprising:
exposing a region of interest to a static magnetic field with sufficient uniformity to carry out a magnetic resonance process;
exposing the region of interest to a RF magnetic field having at least one gradient and detecting magnetic resonance signals emitted from the region of interest; and
generating an image of at least one feature in the region of interest from the received signals.

2. A method according to claim 1, wherein generating an image comprises performing a rotating frame imaging process.

3. A method according to claim 2, wherein the rotating frame imaging process comprises using a RF gradient in a first direction to selectively excite the resonances in a plane (slice) perpendicular to the gradient, then applying a series of refocusing pulses with a RF gradient in a second direction, orthogonal to the first direction, acquiring the resultant spin-echoes in the presence of a gradient in the static field, in a direction perpendicular to the first and second directions, and processing the data to produce an image.

4. A method according to claim 3, wherein the processing the data uses a two dimensional Fourier transform.

5. A method according to claim 3, wherein the processing the data uses a maximum entropy method.

6. A method according to claims 1, 2, 3, 4 or 5, wherein the feature includes a catheter.

7. A method according to claims 1, 2, 3, 4 or 5, further comprising repeatedly exposing the region of interest to said RF magnetic field with the RF magnetic field at different rotational angles with respect to the static magnetic field.

8. An imaging apparatus for carrying out a method according to claim 1, the apparatus comprising:
a magnetic field generating system for generating a static magnetic field in the region of interest with sufficient uniformity to perform a magnetic resonance process;
a RF transmitter and RF receiving system for transmitting a RF magnetic field having at least one gradient into the region of interest and for detecting magnetic resonance signals emitted from the region of interest; and
a system responsive to the received signals to generate an image of at least one feature in the region of interest.

9. The apparatus according to claim 8, wherein the RF transmitting and receiving system generates a RF magnetic field with at least two orthogonal gradients.

10. The apparatus according to claim 8 or claim 9, wherein the RF transmitter and receiver system include a pair of coaxial coils which can be energized in the same sense or in opposite senses so as to produce an RF field which has a gradient or is relatively uniform, respectively.

11. The apparatus according to claim 8 or 9, wherein the system is adapted to carry out a rotating frame imaging process.

12. The apparatus according to claim 10, wherein the system is adapted to carry out a rotating frame imaging process.

13. A method according to claim 6, further comprising repeatedly exposing the region of interest to said RF magnetic field with the RF magnetic field at different rotational angles with respect to the static magnetic field.
